(19) United States
(12) Patent Application Publication
(10) Pub. No.: US 2007/0178061 A1
(43) Pub. Date: Aug. 2, 2007

(54) Therapeutic blended oil composition and method

(76) Inventors: Marco Venturi, Lake Bluff, IL (US); Tara Venturi-Jackson, Lake Bluff, IL (US)

Correspondence Address: GERRY FISCHER, 954 ARNOLD COURT, DES PLAINES, IL, 60016, US

(21) Appl. No.: 11/346,033

(22) Filed: Feb. 2, 2006

(51) Int. Cl. A61K 8/97; A61K 36/87; A61K 36/48; A61K 36/736; A61K 36/72

(52) U.S. Cl. 424/74; 424/735; 424/757; 424/766; 424/769

(57) ABSTRACT

A therapeutic blended oil composition is provided that is useful as a topical application to be absorbed into skin, protect the skin from sun without inducing acne, reduce skin wrinkles lines and dryness. The therapeutic oil composition is created by mixing grape seed oil, soy bean oil, sweet almond oil, sea buckthom seed oil and essential oils. The volume of each constituent added can be modified according to the desired treatment. The composition may be applied topically to absorb into the skin to aid skin tone, tanning, moisturizing, eczema, and bum symptoms. The composition is light oil that does not leave the skin feeling oily and is an effective and safe makeup remover.

### BACKGROUND OF THE INVENTION

### FIELD OF INVENTION

The present invention relates to a therapeutic oil composition and method that is a pleasant smelling topical application to be absorbed into skin, protect the skin from sun without inducing acne, enhance epidermal healing, reduce skin wrinkles, lines and dryness. The therapeutic blended oil composition is created by mixing grape seed oil, soy bean oil, sweet almond oil, sea buckthom seed oil and essential oil. The botanical names of these oils are vitis vinifera, soja hispida, prunus dulcis, hippophae rhamnoides, and essential oil, respectively. The volume of each constituent added can be modified according to the desired treatment. The composition may be applied topically to absorb into the skin to aid skin tone, tanning, moisturizing, eczema, and bum symptoms. The composition is a light oil that does not leave the skin feeling oily and is an effective and safe makeup remover.

Accordingly, there is a need for a therapeutic blended oil composition which can be used for therapy in treating the skin. The present invention fulfills this need and provides other related advantages.

### SUMMARY OF INVENTION

The invention is a therapeutic blended oil composition and its method of production using the oils of vitis vinifera, soja hispida, prunus dulcis, hippophae rhamnoides, and essential oil or grape seed oil, soy bean oil, sweet almond oil, sea buckthorn seed oil and essential oil respectively.

The foregoing and other features and advantages of the invention are apparent from the following detailed description of the exemplary embodiments. Although several embodiments have been described in detail for purposes of illustration, various modifications may be made without departing from the scope of the invention. Accordingly, the detailed description is merely illustrative of the invention rather than limiting, the scope of the invention being defined by the appended daims and equivalents thereof.

### DETAILED DESCRIPTION OF THE INVENTION

The following includes a description of the individual component oils, the preferred extraction method, and the advantageous properties based on past use and history.

Grape seed oil, having the botanical name of vitis vinifera may be used as base oil for creams and lotions. The preferred method of extracting the grape seed oil from the seed is a cold press, non-solvent extraction.

After the pressing of the grapes for wine production, the pomade consisting of the grape skins, stems and seeds is collected and the seeds are separated. The grape seeds are dried before the oil is pressed from them and the oil is bottled. The process used to extract the oil from the seed is a cold-press, no solvent extraction method. During this process, the seeds are dried before the oil is pressed from them and temperatures are kept low to preserve the valuable nutrients contained in grape seed oil. The temperature is kept low to preserve the valuable nutrients of the grape seed oil.

It has been carrier oil used by massage therapists because of its light and satin like finish. Grape seed oil has high levels of linoleic acid, an essential fatty acid and is considered more potent an antioxidant than Vitamins C & E. Grape seed oil is golden yellow with a hearty green tone, has astringent qualities and is useful on oily or acne prone skin. Grape seed oil is odorless and is one of the lightest carrier oils, easily absorbed into the skin. These properties make it a preferred choice for use in massage therapy, cosmetic manufacturing, and aromatherapy.

Grape seed oil is a preferred cosmetic ingredient for damaged and stressed tissues. Its regenerative and restructuring virtues allow control of skin moisturizing. Grape seed oil may help skin retain the normal structure of epithelium cells and nerve cells by supporting the cell membranes. It may be especially effective for repair of the skin around the eyes. When used as a skin moisturizer, grape seed oil reduces the look of stretch marks. Expectant mothers using a grape seed oil based skin moisturizer may have little to no skin damage and stretch marks.

Grape seed oil is the byproduct of wine production. It is made from grape seeds of various varieties of Vitis Vinifera grapes, which are contained in pomace. Pomace is the solid leftover after the juice has been extracted or expressed from the grapes during wine production. Extracting grape seed oil is more difficult than other oils because the grape seed itself is small, has a hard shell, and contains only a small amount of oil. Because the Grape seed has such a hard shell, the natural oil and nutrients are well protected against chemicals such as agricultural sprays and/or additives, thus providing naturally pure oil. Grape seed oil may be synergistically blended with oils to create a powerful antiinkle, dermal enhancing, therapeutic serum that is readily metabolized by your skin.

Grape seed oil is rich in antioxidants and its benefits include, but are not limited to enhancing the absorption of other oils, reducing free radical damage with regular use, metabolizing rapidly by the skin, leaving no greasy residue, diminishing the suns damaging effects on skin, soothing and healing of the skin, having astringent qualities that help to tighten and tone the skin and may help the skin retain the normal structure of epithelium cells and nerve cells via supporting the cell membranes.

Several uses for grape seed oil include massage oil, body hygiene cream, hand cream, lip balm, sunburn repair lotion, hair products, marinade, deep frying, flavored oil, baking and salad dressing.

Soybean oil, having the botanical name of soja hispida is the natural oil extracted from whole soybeans. A preferred method of extraction is expeller pressed, partially refined.

Soybean oil that is expeller pressed and partially or physically refined is processed without the use of chemicals. The oil has stability similar to partially hydrogenated oils without the trans or saturated fats. This method of extraction produces soybean oil that is liquid at room temperature. Liquid expeller pressed and partially refined soybean oils are preferred for commercial blending. Expeller pressed and partial refined soybean oil extraction does not remove any of the naturally occurring antioxidants of the oil. These antioxidants may provide oil stability while the process may stabilize the antioxidants of the soybean oil. Further, expeller pressed and partially refined soybean oil does not use chemical solvents or refining caustics, rather, the extraction uses heat and steam during the extrusion stage to strip free fatty acids.

Soybean oil is high in natural source lecitihin, sterolins, and vitamin E. Two of the polyunsaturated fatty acids in soybean oil include two essential fatty acids, which are not produced in the body, linoleic and linolenic. Linoleic and linolenic acids foster the body's absorption of vital nutrients required for human health. These properties make it a preferred choice for products which are being created for outer epidermal healing. Soybean oil is readily absorbed into the skin, leaving the skin with a smooth sensation.

Soybean oil benefits include, but are not limited to that it is rich in antioxidants, abundant in soy lipids beneficial for regeneration of skin cell, it improves skin texture and reduces water loss, it contains a high content of phytosterols that may increase collagen production, it does not clog pores, it is easily metabolized by your skin and leaves a smooth sensation.

Several uses for soybean oil in the food industry include mayonnaise, non-dairy creamer, salad dressing, margarine, sandwich spread, whipped topping, and snack food. Whole soybeans are high in vitamin E, and though processing removes over 30% of the vitamin E, the processed soybean oil is still considered to be a good source of this important vitamin.

The berries of sea buckthom having the botanical name of hippophae rhamnoides are rich in vitamins and nutrients and clinical trials and scientific studies conducted confirm the medicinal value of Sea Buckthom. The preferred method of extracting the sea buckthom oil is CO2 pressed.

CO2 method of extraction is achieved by using cold CO2 which is in the transition point between liquid and gas. This method of extraction removes much of the plant solid material as well as the oil to forms a waxy or tar like substance which is preferred for cosmetics. Refining these extracts further provides the oils desired. The refined oil is frequently referred to as select.

Sea buckthom berries contain nutrients and vitamins in their seeds including vitamins A, K, E, C, B1, B2, folic acid, essential fatty acids, amino acids, and minerals. Herbal remedies made of sea buckthom are used for the treatment of diseases of skin, to promote the recuperation of skin injuries and support the healing of skin. Sea buckthorn oil extract may be beneficial to skin and facial care by restoring and nourishing healthy skin.

Sea buckthom oil is a rich source of linoleic and linolenic acid and has antioxidant properties due to high phytochemical content, including flavonoids, carotenoids and phytosterols. Medicinally, sea buckthorn oil is used to promote the regeneration of the skin and mucous membranes as well as the recovery of various skin conditions, including eczema, bums, healing wounds, skin damaging effects of sun, therapeutic radiation treatment and cosmetic laser surgery. Cosmetics and skin care products containing sea buckthom rejuvenate, restore and combat the aging of skin.

Sea buckthom oil is beneficial in skin care. The oil has antioxidants and essential fatty acids that may help reverse damaging effects of sun radiation, block UV ray damage, promote the skin's natural restorative processes, reduce inflammation and minimize long term effects of sun exposure such as pigmentation spots, wrinkles and dryness. Sea buckthom oil helps combat acute sunburns and provides long term anti-inflammatory, revitalizing and restoring action.

Sea buckthom oil benefits include, but are not limited to being properly absorbed and quickly assimilated into the skin, containing vitamin C that is essential for reestablishing new collagen growth, containing flavonoids with antioxidant properties, is well tolerated by most skin types, combats acute sunbums, providing long-term anti-inflammatory, restorative, and revitalizing action, reversing negative effects of intense sun radiation, preventing the development of acute sunburn and providing protection against oxidative and free radical damage.

Sea buckthom oil may have an unpleasant odor. A preferred method of removing odor from sea buckthom seed oil where circumstances require high volumes of sea buckthom seed oil to be deodorized is a mechanical aeration process implemented where filtered air is injected into 55 gallons of sea buckthom oil at the rate of 100 cubic feet per minute for approx. four hours. Another preferred method for removing this odor is by letting the oil breathe. The breathing process is preferably applied prior to mixing or blending with other oils, but may also be done at the end of the process. A preferred method of sea buckthom oil breathing process requires approximately one hour and 1300 square inches of surface area per gallon of oil.

Sweet almond oil having the botanical name of prunus dulcis is obtained from the dried kernel of the seeds of the almond tree. This oil has been traditionally used by massage therapists to lubricate the skin during a massage and is considered an effective emollient or soothing agent. The preferred method of extraction is cold pressed, oil extracted by expressing the seeds without implementing heat.

Sweet almond oil benefits include, but are not limited to being an excellent emollient for most skin types, having minerals, protein, naturally occurring vitamins and mono and poly-unsaturated fatty acids, it promotes skin cell renewal, it is close to the natural oils found in skin, it softens wrinkles and fine lines, it promotes the natural beauty of skin with a younger looking complexion, it is a natural moisturizer, it is beneficial to dry or chapped skin, it is high in essential fatty acids, it does not clog pores, it enhances skin pigmentation when tanning, it helps the skin balance its loss and absorption of moisture and may relieve muscular aches and pains.

Sweet almond oil is frequently used as base oil in aromatherapy and may be used as an emollient on dry or chapped skin. Sweet almond oil consists of vitamins, linolenic acid, oleic acid, myristic acid and palmitic acid. The oil is almost completely odorless and can be used as a gentle makeup remover or aromatherapy carrier oil.

Essential oil is a hydrophobic liquid containing aromatic compounds extracted from plants. The oil is a highly volatile substance isolated from an odoriferous plant of a single botanical species and is labeled essential because they represent the very essence of its odor and flavor. Essential oil preferred production method is by expression, distillation, or solvent extraction. Essential oils are used in cosmetics, perfume, aromatherapy, incense and medicine. Essential oil is also known as ethereal and volatile oil or the oil of the raw plant material from which it was extracted.

A preferred essential oil processing method is by distilling the raw plant material over water, the volatile compounds being vaporized as the water is heated. The vapor may be directed to a cooling tank, the steam condensed, and the lighter than water essential oil floats to the top.

Essential oils are aromatic oils obtained by the hydro-distillation of botanicals. Several parts of the plants may be used to obtain essential oils, including the flowers, leaves, seeds, roots, stems, peels, wood or bark. Certain cold-pressed oils, such as the oils from various citrus peels, are also considered to be essential oils.

A list of preferred sources of essential oil includes, but is not limited to allspice berry, almond seed, anise seed, basil leaf, bay leaf, bergamot peel, camphor wood, cedar wood, celery seed, chamomile flower, cassia bark, citronella, cinnamon leaf or bark, clary sage flowering tops, clove bud, common sage leaf, cumin seed, eucalyptus leaf, frankincense resin, geranium flower, ginger rhizome, grapefruit peel, hyssop flower, jasmine flower, juniper berry, lavender flower, lemon peel, lemon grass leaf, manuka flower, marjoram flower, melaleuca leaves, myrrh resin, nutmeg oil seed, orange peel, flower or leaf, oregano leaf, patchouli leaf, peppermint leaf, pine leaf, rose flower, rosemary leaf, rosewood wood, sandalwood wood, spearmint leaf, tangerine peel, tea tree leaf, thyme leaf, wintergreen leaf and ylang-ylang flower.

A preferred method for blending the oils includes combining the oils in a container and mixing the oils at a preferred temperature between approximately sixty five (65) and seventy (70) degrees Fahrenheit.

Topically antioxidants such as the blended oil composition of this invention may be applied on a regular basis. Millions of dead skin cells are replaced each day with new skin cells. The skin renewal process is constantly under attack from free radical exposure. As the skin cells are renewed, free radicals bond molecularly with the new skin cells causing them to renew in a mutilated state. As each day passes these mutilated skin cells develop and appear as aged and wrinkled skin. A preferred outcome is that when antioxidants are applied to the skin on a regular and consistent basis, the skin cells will reproduce as normal and healthy as opposed to mutilated. When the dead, mutilated or deformed skin cells are replaced by new healthy skin cells that have been regularly treated with antioxidants, it is just a matter of time before the skin begins to take on its original healthy and youthful appearance.

What is claimed is:

1. A method for creating a therapeutic blended oil composition, comprising the step of:
mixing oils of vitis vinifera, soja hispida, prunus dulcis, hippophae rhamnoides, and essential oil.

2. The method of claim 1, wherein the oils of vitis vinifera, soja hispida, prunus dulcis, hippophae rhamnoides, and essential oil are each between approximately 0.5% and 95% by volume of the total composition volume.

3. The method of claim 2, wherein the mixing step of oils of vitis vinifera, soja hispida, prunus dulcis, hippophae rhamnoides, and essential oil includes the step of storing the mixture at a temperature of approximately between 60 degree F. to 75 degree F.

4. The method of claim 2, wherein the mixing step of oils of vitis vinifera, soja hispida, prunus dulcis, hippophae rhamnoides, and essential oil includes the step of allowing the hippophae rhamnoides to breathe for a predetermined time period prior to mixing.

5. The method of claim 4, wherein the predetermined time period is approximately one hour per gallon.

6. The method of claim 2, wherein the oil of vitis vinifera is approximately 45 to 95%, soja hispida is approximately 5 to 55%, prunus dulcis is approximately 5 to 55%, hippophae rhamnoides is approximately 1 to 55%, and essential oil is approximately 0.5 to 10% by volume of the total composition volume.

7. The method of claim 2, wherein the oil of vitis vinifera is approximately 65%, soja hispida is approximately 16%, prunus dulcis is approximately 16%, hippophae rhamnoides is approximately 2.5%, and essential oil is approximately 0.5% by volume of the total composition volume.

8. The method of claim 2, wherein the oil of vitis vinifera is approximately 88%, soja hispida is approximately 5%, prunus dulcis is approximately 5%, hippophae rhamnoides is approximately 1.5%, and essential oil is approximately 0.5% by volume of the total composition volume.

9. A therapeutic blended oil composition, comprising oils of:
vitis vinifera;
soja hispida;
prunus dulcis;
hippophae rhamnoides; and
essential oil.

10. The composition of claim 9, wherein the oils of vitis vinifera, soja hispida, prunus dulcis, hippophae rhamnoides, and essential oil are each approximately between 0.5% and 95% by volume.

11. The composition of claim 9, wherein the oils of the composition are aged.

12. The composition of claim 11, wherein the oils of the composition are stored at a temperature between approximately 60 degrees Fahrenheit to 75 degrees Fahrenheit.

13. A therapeutic blended oil composition, consisting essentially of:
an aged mixture of oils of vitis vinifera, soja hispida, prunus dulcis, hippophae rhamnoides, and essential oil each approximately between 0.5% and 95% by volume.

14. The composition of daim 13, wherein the oil of vitis vinifera is approximately 45 to 95%, soja hispida is approximately 5 to 55%, prunus dulcis is approximately 5 to 55%, hippophae rhamnoides is approximately 1 to 55%, and essential oil is approximately 0.5 to 10% by volume of the total composition volume.

15. The composition of daim 13, wherein the oil of vitis vinifera is approximately 65%, soja hispida is approximately 16%, prunus dulcis is approximately 16%, hippophae rhamnoides is approximately 2.5%, and essential oil is approximately 0.5% by volume of the total composition volume.

16. The composition of daim 13, wherein the oil of vitis vinifera is approximately 88%, soja hispida is approximately 5%, prunus dulcis is approximately 5%, hippophae rhamnoides is approximately 1.5%, and essential oil is approximately 0.5% by volume of the total composition volume.
